(19) United States
(12) Patent Application Publication
(10) Pub. No.: US 2003/0054524 A1
(43) Pub. Date: Mar. 20, 2003

(54) Method of increasing the fatty acid content in plant seeds

(76) Inventors: Friedrich Spener, Muenster (DE); Burkhardt Siegfried Schutt, Rottenburg/Neckar (DE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/013,803

(22) Filed: Dec. 10, 2001

Related U.S. Application Data
(63) Continuation of application No. PCT/EP00/05338, filed on Jun. 9, 2000.

(30) Foreign Application Priority Data
Jun. 10, 1999 (DE) 199 26 456.2

(51) Int. Cl. A01H 5/00; C07H 21/04; C12N 9/10; C12N 5/04

(52) U.S. Cl. 435/193; 435/410; 435/320.1; 435/325; 536/23.2; 800/281

(57) ABSTRACT

The invention relates to nucleic acid molecules that encode a protein with the activity of a β-ketoacyl-ACP synthase IV (KASIV) from Cuphea lanceolata, to nucleic acid molecules that encode a protein with the activity of a β-ketoacyl-ACP synthase II (KASII) from Brassica napus, and to nucleic acid molecules that encode a protein with the activity of a β-ketoacyl-ACP synthase I (KASI) from Cuphea lanceolata. The invention further relates to a method of increasing the content of fatty acids, especially of short- and medium-chain fatty acids in triglycerides of plant seeds. The inventive method comprises expressing a protein with the activity of KASII or a protein with the activity of KASIV in transgenic plant seeds.

The present invention relates to nucleic acid molecules encoding a protein with the activity of a β-ketoacyl-ACP synthase IV (KASIV) from Cuphea lanceolata, nucleic acid molecules encoding a protein with the activity of a β-ketoacyl-ACP synthase II (KASII) from Brassica napus and nucleic acid molecules encoding a protein with the activity of a β-ketoacyl-ACP synthase I (KASI) from Cuphea lanceolata. In addition, this invention also relates to methods of increasing the fatty acid content, in particular the short- and medium-chain fatty acids, in triglycerides of plant seeds, including expression of a protein with the activity of a KASII or a protein with the activity of a KASIV in transgenic plant seeds.

Fatty acid biosynthesis and triglyceride biosynthesis can be regarded as separate biosynthesis pathways due to compartmentalization, but as one biosynthesis pathway from the standpoint of the end product. De novo biosynthesis of fatty acids takes place in plastids and is catalyzed by essentially three enzymes or enzyme systems, namely acetyl-CoA-carboxylase, fatty acid synthase and acetyl-ACP-thioesterase. In most organisms, the end products of this reaction sequence are palmitate, stearate and, after desaturation, oleate.

Fatty acid synthase is an enzyme complex consisting of individual enzymes that can be dissociated, the individual enzymes being acetyl-ACP-transacylase, malonyl-ACP-transacylase, β-ketoacyl-ACP-synthases (acyl-malonyl-ACP condensing enzymes), β-ketoacyl-ACP-reductase, 3-hydroxyacyl-ACP-dehydratase and enoyl-ACP-reductase.

The elongation phase of fatty acid synthesis begins with the formation of acetyl-ACP and malonyl-ACP. Acetyl-transacylase and malonyl-transacylase act as catalysts in this reaction. Acetyl-ACP and malonyl-ACP react to form acetoacetyl-ACP, and this condensation reaction is catalyzed by the acyl-malonyl-acetyl condensing enzyme. In the next three steps of fatty acid synthesis, the keto group on the C-3 is reduced to a methylene group, with the acetoacetyl-ACP first being reduced to D-3-hydroxybutyryl-ACP and then crotonyl-ACP being formed from D-3-hydroxybutyryl-ACP by splitting off water. In the last step of the cycle, crotonyl-ACP is reduced to butyryl-ACP, so that the elongation cycle is concluded. In the second round of fatty acid synthesis, butyryl-ACP is condensed with malonyl-ACP to form C₆-β-ketoacyl-ACP. Subsequent reduction, splitting off water and a second reduction convert C₆-β-ketoacyl-ACP to C₆-acyl-ACP, which is made available for a third round of elongation. These elongation cycles continue until C₁₆-acyl-ACP is obtained. This product is no longer a substrate for the condensing enzyme and instead it is hydrolyzed to palmitate and ACP.

Then in the so-called Kennedy pathway, triacylglyceride biosynthesis from glycerin 3-phosphate and fatty acids which are present in the form of an acyl-CoA substrate takes place in the cytoplasm on the endoplasmic reticulum.

The term fatty acid includes saturated or unsaturated short-, medium- or long-chain, linear or branched, even-numbered or odd-numbered fatty acids. Short-chain fatty acids include in general fatty acids having up to six carbon atoms. These include butyric acid, valeric acid and hexanoic acid. The term medium-chain fatty acid includes C₈ through C₁₄ fatty acids, i.e., primarily octanoic acid, capric acid, lauric acid and myristic acid. Finally, the long-chain fatty acids include those with at least 16 carbon atoms, i.e., mainly palmitic acid, stearic acid, oleic acid, linoleic acid and linolenic acid.

Fatty acids which occur in all vegetable and animal fats, mainly in vegetable oils and fish oils, have a variety of uses. For example, a deficiency of essential fatty acids, i.e., fatty acids that cannot be synthesized in the body and therefore must be ingested in the diet, leads to skin changes and growth disorders, which is why fatty acids are used in eczema, psoriasis, bums and the like as well as in cosmetics. In addition, fatty acids and oils are also used in laundry and cleaning products, as detergents, as dye additives, lubricants, processing aids, emulsification aids, hydraulic oils and as carrier oils and vehicles in pharmaceutical and cosmetic products. Natural oils and fats of animal origin (e.g., tallow) and of plant origin (e.g., coconut oil, palm kernel oil or canola oil) are used as renewable raw materials in the field of chemical engineering. The areas for use of vegetable oils have expanded greatly in the last twenty years. With an increase in environmental awareness, environmentally friendly lubricants and hydraulic oils, for example, have been developed. Fats and fatty acids have other applications as foods and food additives, e.g., in parenteral nutrition, as baking aids, in baby food, food for seniors and athletes, in chocolate preparations, cocoa powder and as backing fats, for the production of soaps, creams, ointments, candles, artists' paints and textile dyes, varnishes, heating and lighting means.

One of the goals in plant cultivation is to increase the fatty acid content of seed oils. There is a cultivation goal with respect to industrial rapeseed and alternative production areas for agricultural in production of rapeseed oil with fatty acids of a medium chain length, mainly C₁₂, because these are in high demand for the production of surfactants. In addition to the idea of using vegetable oils as industrial raw materials, there is the possibility of using them as biopropellants.

Therefore, there has been a demand for a supply of fatty acids which can be used industrially, e.g., as basic materials for plasticizers, lubricants, pesticides, surfactants, cosmetics, etc. and/or are valuable in food technology. One possibility of supplying fatty acids is by extraction of the fatty acids from plants which contain especially high levels of these fatty acids. It has so far been possible to increase the medium-chain fatty acid content, for example, only to a limited extent by traditional methods, i.e., by cultivation of plants that produce these fatty acids to an increased extent.

Therefore, one object of this invention is to make available genes or DNA sequences which can be used to improve the oil yield and for production of fatty acids in plants which produce these fatty acids only to a slight extent or not at all. In particular, it is also the object of this invention to make available DNA sequences which are suitable for increasing the medium- and short-chain fatty acid content in plants, in particular plant seeds.

Another object is to provide methods of increasing the fatty acid content, in particular the medium- and short-chain fatty acids in plant seeds.

The features of the independent patent claims achieve these goals.

Advantageous embodiments are defined in the respective subordinate claims.

It has now surprisingly been possible for the first time to assign an exact substrate specificity to the β-ketoacyl-ACP-synthase IV enzyme which is involved in fatty acid synthesis. Accordingly, KAS IV is capable of effectively catalyzing the elongation of acyl-ACP substrates up to a chain length of CIo-ACP, but further elongation takes place only with a comparatively low activity. This observation is used according to this invention to increase the medium-chain fatty acid content in plants.

This invention is thus a method of increasing the medium-chain fatty acid content in plant seeds, comprising the steps:

a) Production of a nucleic acid sequence comprising at least the following components which are aligned in the 5′-3′ orientation: a promoter which is active in plants, especially in embryonal tissue, at least one nucleic acid sequence encoding a protein with the activity of a β-ketoacyl-ACP-synthase IV or an active fragment thereof and optionally a termination signal for termination of transcription and addition of a poly-A tail to the corresponding transcript and optionally DNA sequences derived therefrom;

b) transferring nucleic acid sequences from a) to plant cells and

c) optionally regenerating completely transformed plants and reproducing the plants, if desired.

In a preferred embodiment, the KAS IV sequences are transferred together with a suitable thioesterase to synthesize the largest possible amounts of medium-chain fatty acids. There are already known thioesterase sequences, e.g., those from: International Patent WO 95/06740, WO 92/11373, WO 92/20236 and WO 91/16421.

In addition, it has surprisingly been found that plant enzymes with the activity of a ketoacyl-ACP-synthase II do not synthesize only long-chain fatty acids, as was previously assumed, i.e., using C₁₄- and C₁₆-acyl-ACP substrates, but instead they also have a specificity for C₄- and C₆-substrates. This means that a method of increasing the short-chain fatty acid content in plant seeds, comprising the following steps:

a) Producing a nucleic acid sequence comprising at least the following components, which are aligned in 5=40 -3′ orientation: a promoter which is active in plants, especially in embryonal tissue, at least one nucleic acid sequence encoding a protein with the activity of a β-ketoacyl-ACP-synthase II or an active fragment thereof and optionally a termination signal for termination of transcription and addition of a poly-A tail to the corresponding transcript, plus optionally DNA sequences derived therefrom;

b) transferring the nucleic acid sequence from a) to plant cells, and

c) optionally regenerating completely transformed plants and reproducing the plants, if desired.

In a preferred embodiment, in addition to KAS II sequences, DNA constructs which guarantee suppression of endogenous KAS I sequences are also transferred, e.g., antisense or co-suppression constructs against KAS I. Since endogenous KAS I activity naturally causes elongation of short-chain substrates to medium-chain fatty acids, suppressing endogenous KAS I activity is an efficient method of supplying and accumulating short-chain fatty acids.

In a preferred embodiment, the KAS sequences according to this invention are expressed under the control of seed-specific regulatory elements, in particular promoters, in plant cells. Thus, the DNA sequences according to this invention are present in combination with promoters that are especially active in embryonal tissue. Examples of such promoters include the USP promoter (Baumlein et al. 1991, Mol. Gen. Genet. 225:459467), the Hordein promoter (Brandt et al. 1985, Carlsberg Res. Commun. 50: 333-345) and the napin promoter, the ACP promoter and the FatB3 and FatB4 promoters, with which those skilled in the field of plant molecular biology are very familiar.

The nucleic acid sequences according to this invention can be supplemented by enhancer sequences or other regulatory sequences. The regulatory sequences also include, for example, signal sequences which ensure the transport of the gene product to a certain compartment.

The present invention also relates to nucleic acid molecules which contain the nucleic acid sequences according to this invention or parts thereof, i.e., also vectors, in particular plasmids, cosmids, viruses, bacteriophages and other vectors which are conventionally used in genetic engineering and can optionally be used for transfer of the nucleic acid molecules according to this invention to plants or plant cells.

The plants which are transformed with the nucleic acid molecules according to this invention and in which an altered amount of fatty acids is synthesized because of the introduction of such a molecule may include in principle any desired plants, preferably monocotyledonous or dicotyledonous crop plants and especially preferably an oil plant. Examples include in particular canola, sunflower, soybeans, peanuts, coconut, rapeseed, cotton and oil palms. Other plants which can be used in the production of fats and fatty acids or as foodstuffs having an increased fatty acid content include flax, poppy, olive, cocoa, corn, almond, sesame, mustard and ricinus.

Furthermore, this invention also relates to replication material from plants according to this invention, e.g., seeds, fruit, seedlings, tubers, root stock, etc., as well as parts of these plants such as protoplasts, plant cells and callus.

In a preferred embodiment, the KAS IV DNA sequences are DNA sequences isolated from Cuphea lanceolata.

The KAS II sequences are preferably sequences isolated from Brassica napus.

Various methods have been proposed for production of the plants according to this invention. First, plants or plant cells can be modified with the help of traditional methods of transformation in genetic engineering such that the new nucleic acid molecules are integrated into the plant genome, i.e., stable transformants are created. Secondly, a nucleic acid molecule according to this invention, whose presence and optional expression in the plant cell produce an altered fatty acid content, may be present in the plant cell or in the plant itself as a self-replicating system.

A large number of cloning vectors are available for preparation for introduction of foreign genes into higher plants, which contain replication signals for Escherichia coli and a marker gene for selection of transformed bacterial cells. Examples of such vectors include pBR322, pUC series, M13mp series, pACYC184, etc. the desired sequence can be introduced into the vector in a suitable restriction cleavage site. The resulting plasmid is then used for transformation of E. coli cells. Transformed E. coli cells are cultured in a suitable medium and then harvested and lysed, and the plasmid is recovered. In general, restriction analyses, gel electrophoresis methods and other methods of biochemistry and molecular biology are used as analytical methods to characterize the plasmid DNA thus obtained. After each manipulation, the plasmid DNA can be cleaved and the DNA fragments thus obtained can be combined with other DNA sequences.

A number of known techniques are available for introduction of DNA into a plant host cell, and those skilled in the art can easily determine the most suitable method in each case. These techniques include transformation of plant cells with T-DNA using Agrobacterium tumefaciens or Agrobacterium rhizogenes as the means of transformation, fusion of protoplasts, direct gene transfer of isolated DNA in protoplasts, electroporation of DNA, introduction of DNA by means of the biolistic method as well as other possibilities.

In injection and electroporation of DNA in plant cells, there are no special requirements of the plasmids used. The same thing is also true of direct gene transfer. Simple plasmids such as pUC derivatives may be used. However, if entire plants are to be regenerated from such transformed cells, the presence of a selectable marker gene is necessary. Those skilled in the art will know of gene selection markers, and it would not be any problem for them to select a suitable marker.

Depending on the method of introduction of desired genes into the plant cell, other DNA sequences may also be necessary. For example, if the Ti or Ri plasmid is used for transformation of the plant cell, then at least the right border but often the right and left borders of the T-DNA contained in the Ti and Ri plasmids must often be linked as the flank area to the genes to be introduced.

If Agrobacteria are used for the transformation, the DNA to be introduced must be cloned in special plasmids, namely either in an intermediate vector or a binary vector. Intermediate vectors can be integrated into the Ti or Ri plasmid of Agrobacteria by homologous recombination on the basis of sequences which are homologous with sequences in the T-DNA. It also contains the vir region which is necessary for transfer of the T-DNA. Intermediate vectors cannot replicate in Agrobacteria. The intermediate vector can be transferred to Agrobacterium tumefaciens by means of a helper plasmid (conjugation). Binary vectors can replicate in both E. coli and Agrobacteria. They contain a selection marker gene and a linker or polylinker which is bordered by the right and left T-DNA bordering regions. They can be transformed directly in Agrobacteria. The Agrobacterium which serves as the host cell should contain a plasmid which has a vir region. The vir region is necessary for transfer of T-DNA into the plant cell. Additional T-DNA may be present. Agrobacterium transformed in this way is used for transformation of plant cells.

The use of T-DNA for transformation of plant cells has been researched extensively and has been described adequately in well-known review articles and handbooks on plant transformation.

For transfer of the DNA to the plant cell, plant explantates may be cultured with Agrobacterium tumefaciens or Agrobacterium rhizogenes. Entire plants can be regenerated again from the infected plant material (e.g., leaf fragments, stem segments, roots as well as protoplasts or suspension-cultured plant cells) in a suitable medium which may contain antibiotics or biocides for selection of transformed cells. The plants are regenerated according to conventional regeneration methods using known culture media. The resulting plants can then be tested for the presence of the DNA introduced. Other possibilities for introduction of foreign DNA using the biolistic method or by protoplast transformation are also known and have been described repeatedly.

Once the DNA thus introduced has been integrated into the genome of the plant cell, it is usually stable there and also remains in the progeny of the cell transformed originally. It normally contains a selection marker which imparts to the transformed plant cells a resistance to a biocide or an antibiotic such as kanamycin, G418, bleomycin, hydromycin, methotrexate, glyphosate, streptomycin, sulfonyl urea, gentamycin or phosphinothricin and the like. Therefore, the individually selected marker should permit selection of transformed cells with respect to cells lacking the introduced DNA.

The transformed cells grow in the usual way within the plant. The resulting plants can be cultivated normally and can be crossed with plants having the same transformed genetic trait or different genetic traits. The resulting hybrid individuals have the corresponding phenotypic properties. Seeds can be obtained from the plant cells.

Two or more generations should be cultivated to ensure that the phenotypic feature is retained as a stable trait and is inherited. Seeds should also be harvested to ensure that the corresponding phenotype or other traits are preserved.

Likewise, by the usual methods it is possible to determine transgenic lines which are homozygous for the new nucleic acid molecules and whose phenotypic behavior has been investigated with respect to an altered fatty acid content and compared with that of hemizygous lines.

The proteins according to this invention can be expressed with KAS II or KAS IV activity with the help of traditional methods of biochemistry and molecular biology. Those skilled in the art are familiar with these techniques and are capable of selecting with no problem a suitable detection method such as a Northern Blot analysis for detection of KAS-specific RNA or for determining the amount of accumulation of KAS-specific RNA, a Southern Blot analysis for identification of DNA sequences encoding KAS II and KAS IV or a Western Blot analysis for detection of the protein encoding the DNA sequences according to this invention, i.e., KAS II or KAS IV. The enzymatic activity of KAS II or KAS IV can be detected on the basis of a fatty acid pattern or an enzyme assay, e.g., as described in the following examples.

In most cases, an enrichment with certain fatty acids in plants, in particular in the seeds or fruit, is desirable, but it may also be desirable to reduce the amount of certain fatty acids, e.g., for dietary reasons. In this case, the sequences and methods according to this invention can be used to suppress the synthesis of medium- and short-chain fatty acids in plants. The methods that can be used in this case, in particular the antisense technique and the co-suppression strategy, will be familiar to those skilled in the art in the field of plant biotechnology.

This invention is based on the successful isolation of novel KAS II and KAS IV clones and the assignment of concrete substrate specificities, performed successfully here for the first time, as described in the following examples.

The following examples are presented to illustrate this invention.

### EXAMPLES

### Example 1

### Cloning a cDNA Clone for KAS II From Brassica Napus

Whole RNA was isolated from embryos of developing seeds of Brassica napus according to the method of Voeltz et al. (1994) Plant Physiol. 106:785-786, and MRNA was extracted using oligo-dT-cellulose (Qiagen, Hilden, Germany); cDNA pools were prepared from MRNA preparations by reverse transcription with an oligo-dT adapter primer (5′-AACTGGAAGAATTCGCGGCCGCAGGAAT₁₈-3′). Based on preserved regions of KAS II encoding genes from H. vulgare (Wissenbach et al. (1994) Plant Physiol. 106:1711-1712), R. communis (Knauf and Thompson (1996) U.S. Pat. No. 5,510,255) and B. rapa (Knauf and Thompson (1996) U.S. Pat. No. 5,510,255), degenerated oligonucleotides were constructed to produce PCR products of both cDNA templates. Oligonucleotides ,,5kas2″ (5′-ATGGGNGGCAGTGAAGGTNTT-3′) and ,,3kas2″ (5′-GTNGANGTNGCATGNGCATT-3′) were constructed according to the amino acid sequences MGGMKVF and NAHATST (horizontal arrows in FIG. 1). PCR products produced using these oligonucleotide primers were sequenced and then the following strategies were pursued.

For cloning a KAS II cDNA from Brassica napus (bnKASII) encoding the mature protein, semi-specific oligonucleotides were constructed with a 5′-NdeI restriction cleavage site based on the known sequences of B. rapa KAS 11 (5′ primer: 5′-CATATGGARAARGAYGCNATGGT-3′, 3′ primer: 5′-TCANTTGTANGGNGCRAAAA-3′), and the resulting bnKASIIa cDNA was cloned in the NdeI restriction cleavage site of the pETI 5b expression vector (Novagen, Madison Wis., USA).

Two different clones were obtained, bnKASIIa and bnKASIIb, whose derived amino acid sequences had 97.4% identity (see FIG. 1). The DNA sequence of the cDNA clone bnKASIIa is shown in SEQ ID no. 3, and the DNA sequence of the cDNA clone bnKASIlb is shown in SEQ ID no. 5. The derived amino acid sequences are shown in SEQ ID no. 4 and SEQ ID no. 6. The clone bnKASIIb has gaps in positions 10-14 and 146-150, the first gap also being in the B. rapa sequence, and the second gap being responsible for the loss of the peptide PFCNP, a pattern that is present in all other KASI sequences known so far. This pattern is essential for formation of the potential substrate binding pocket for E. coli KAS II (* in FIG. 1) which surrounds the cysteine of the active site (Huang et al. (1998) Embo J. 17:1183-1191).

Clone bnKASIIa encodes a polypeptide of 427 amino acids which have an identity of 65% with enzymes of the KASI type of Rhizinus communis (L13242), Arabidopsis thaliana (U24177) and Hordeum vulgare (M7604 10) and an identity of more than 85% with enzymes of the presumed KASII type of R. communis (Knauf and Thompson, loc. cit.) and H. vulgare (Z34268 and Z342690.

### Example 2

### Cloning a cDNA for KASIV From Cuphea Lanceolata

PCR products were prepared as described in Example 1.

For cloning full length cDNA of C. lanceolata, new specific oligonucleotides were constructed according to the sequence information of the first PCR fragment as described above, so that 3′- and 5′-RACE (rapid amplification of cDNA ends) could be performed with them. For production of recombinant protein, the clKASIV cDNA encoding mature protein was constructed by introducing an NdeI restriction cleavage site on methionine¹⁰⁶ by using the PCR technique (see FIG. 1). Modified cDNa was inserted into the NdeI cleavage site of the His-tag expression vector pET15b. All PCR reactions were performed using Pfu DNA polymerase (Stratagene, Heidelberg, Germany).

Sequence comparisons of all the resulting clones showed that the first 435 base pairs and the last 816 base pairs of the cDNA fragment (clKASIVm) that encode the mature protein were identical with the corresponding pats of a 5′-RACE fragment or a 3′-RACE fragment, which is why a theoretical full length cDNA referred to as clKASIV (SEQ ID no. 1) was derived (FIG. 2). This clKASIV cDNA includes a 5′-untranslated region with 33 base pairs, a coding region with 1617 base pairs and a 3′-untranslated region comprising 383 base pairs. The derived amino acid sequence of the clKASIV for the mature protein had an identity of more than 94% with the recently published KASIV sequences of C. wrightii (Slabaugh et al. (1998) Plant J 13: 611-620, C. hookeriana and C. pulcherrima (Dehesh et al. (1998) Plant J. 15: 383-390). The identity with sequences of the KASII type and with bnKASIIa is approximately 85%, whereas the identity with sequences of the KASI tpe is approximately 65%.

### Example 3

### Expression and purification of Recombinant KASII and KASIV Enzymes

Freshly transformed E. coli BL21 (DE3) cells were cultured with 50 g/mL ampicillin at 25EC in 2 liters of TB medium. At a cell density of 0.7 to 0.8 OD₆₀₀ expression of the recombinant proteins was induced by adding isopropyl thiogalactoside up to a final concentration of 20 μM, and the cell growth was continued for one more hour. The cells were harvested by centrifugation and stored overnight at −20 ° C.

The cells were lysed for 30 minutes on ice in 20 ml of the following solution: 5 mM sodium phosphate, pH 7.6, 10% (v/v) glycerol, 500 mM sodium chloride, 10 mM imidazole, 0.1 mM phenylmethylsulfonyl fluoride, 100 μg, 100 gg/nL lysozyme and 2.5 U/mL benzonase. The remaining cells were broken up by sonification (3×10 s), and the entire soluble fraction was loaded onto an Ni-NTA Superflow column (5 mL Qiagen, Hilden, Germany). Nonspecifically bound proteins were removed by washing with 40 mL of 50 mM sodium phosphate, pH 7.6, containing 500 mM sodium chloride, 10% (v/v) glycerol and 50 mM imidazole. In a second washing step, the column was treated with 20 mL of 50 mM sodium phosphate, pH 7.6, containing 10% (v/v) glycerol and 50 mM imidazole to remove the sodium chloride. Finally, the recombinant enzymes were eluted with the same buffer, although it contained 250 mM imidazole for this step. The fractions were stored at −70° C. until being used.

The yield was approx. 250 μg soluble recombinant enzyme per liter of culture. SDS-PAGE showed that the affinity-purified enzymes KASII and KASIV were essentially free of protein contamination. The recombinant enzymes including the N-terminal fusion His-tag, have the predicted molecular weights of 48.0 kDa (bnKAS1ia) and 48.5 kDa (clKASIV), which is in good agreement with the molecular weight of 47 kDa in SDS-PAGE. The authenticity of both proteins was verified by antibody staining with anti-His-tag antibodies.

### Example 4

### Producing Acyl-ACP Substrates

ACP of E. coli was obtained from Sigma (Deisenhofen, Germany) and was purified by anion exchange FPLC on Mono Q, as described by Kopka et al. (1993) Planta 191: 102-111. C₆ through C₁₆ acyl-ACPs were synthesized enzymatically from E. coli ACP using an acyl-ACP synthase from Vibrio harveyi (Shen et al. (1992) Anal. Biochem. 204:34-39). Butyryl-ACP was synthesized chemically according to Cronan and Klages (1981) Proc. Natl. Acad. Sci. USA 78:5440-5444) and was purified further according to Briick et al. (1996) Planta 198:271-278. The purity and concentration of the acyl-ACP stock solutions was determined by conformationally sensitive gel electrophoresis in 20% acrylamide gels containing 2.5 M urea, followed by visualization with Coomassie Blue and densitometric quantification, using purified ACP of a known concentration as the standard. Malonyl-ACP was synthesized enzymatically from ACP and malonyl-CoA using a partially purified malonyl-CoA:ACP-transacylase (MAT) from C. lanceolata seeds (Bruck et al. (1994) J Plant Physiol. 143: 550-555). The reaction mixture (0.5 mL) contained 100 mM sodium phosphate, pH 7.6, 40 μM purified ACP, 80 μM [2-¹⁴C]-malonyl- CoA (0.74 MBq/mmol), 150 FL MAT preparation (corresponding to 0.22 nkat) and 2 mM dithiothreitol (DTT). For complete reduction, ACP was preincubated with DTT for 15 minutes at 37 ° C. before adding the other ingredients. The reaction was allowed to continue for ten minutes at 37EC and was stopped by adding 55 FL of 100% (w/v) trichloroacetic acid (TCA). After incubating on ice for at least ten minutes, the mixture was centrifuged (16,000 g's, 5 minutes, 4 ° C.) and the supernatant containing the unreacted malonyl-CoA was removed and discarded. The precipitate was washed with 200 μl of 1% (w/v) TCA, centrifuged as described above and dissolved in 50 mM 2-(N-morpholino)ethanesulfonic acid, pH 6.8, and stored in aliquots at −20 ° C. The concentration of the [2-¹⁴C]-malonyl-ACP preparation was determined on the basis of liquid scintillation spectrometry data.

### Example 5

### Enzyme Assay

The substrate specificities of the recombinant KASII and KASIV enzymes was investigated by incorporating radioactivity of [2-1⁴C]-malonyl-ACP into the condensation products. The batch (50 gL) contained 100 mM sodium phosphate, pH 7.6, 10 gM acyl-ACP with a specific chain length, 7.5 μM [2-¹⁴C]-malonyl-ACP (0.74 MBq/mmol), 2 mM NADPH, 2 mM DTT, 0.6 Fkat of affinity-purified recombinant GST-β-ketoacyl-ACP-reductase fusion protein of C. lanceolata (Klein et al. (1992) Mol. Gen. Genet. 233:122-128) and 2 μg of the recombinant KASII/IV preparation. The β-hydroxyacyl-ACPs that were synthesized were precipitated, washed and dissolved as described by Winter et al. (1997) Biochem. J 321:313-318 and then separated by a 2.5 M urea-PAGE. After transfer to an Immobilon P membrane by electroblotting at 0.8 mA/cm for one hour, the reaction products were visualized by autoradiography after five-day exposure on an x-ray film (Hyperfilm MP, Amersham, Braunschweig, Germany).

In the assays, saturated acyl-ACP (C₄ through C₁₆) was added to the reaction mixture together with [2- ⁴C]-malonyl-ACP and was incubated for ten minutes. Incorporation of the radioactivity from [2- ⁴C]-malonyl-ACP into the O-ketoacyl-ACP product, which was reduced to β-hydroxyacyl-ACP for the analysis, was determined. The results show various traits for two phylogenetically closely related condensation enzymes. Although the elongation of C₁₄- and C₁₆-ACPs could be observed for bnKASIIa catalysis, as expected for plants that produce long-chain fatty acids, elongation of short-chain acyl-ACPs up to C₆ was also observed (see FIG. 3A).

Investigation of clKASIV catalysis revealed a short-chain-specific condensation activity and, in contrast with KASIIa, a subsequent medium-chain-specific condensation activity up to CIO (see FIG. 3B). In addition, the sensitivity of clKASIV to cerulenin was higher (IC₅₀=20 μM) in comparison with bnKASIla but was nevertheless much lower than the sensitivity known for enzymes of the KASI type, which are already completely inactivated in the presence of 5 μM cerulenin (Shimakata and Stumpf (1982) Proc. Natl. Acad. Sci USA 79:5808-5812). Cerulenin is assumed to be a substrate analog for C₁₂-ACP (Morisaki et al. (1993) Eur. J. Biochem. 211:111-115), so it can be demonstrated reproducibly that the specificity of KASWV for medium-chain acyl-ACPs makes this enzyme more sensitive to cerulenin than KASII.

In summary, it has thus been demonstrated here for the first time that both KASII and KASIV are capable of elongating short-chain acyl-ACP products (C₄ and C₆), but only KASIV catalyzes the elongation of acyl-ACP of C₈-C₁₂. On the other hand, only KASII has a high condensation activity for the substrates C₁₄-ACP and C₁₆-ACP, while KASIV lacks these activities.

### DESCRIPTION OF THE FIGURES

FIG. 1: Alignment of the amino acid sequences of bnKASIIa, bnKASIlb and clKASIV, derived from the respective nucleotide sequences. The amino acids used for the design of the degenerated primers Skas2 and 3kas2 are marked by horizontal arrows. A vertical arrow marks the presumed start of the mature clKAS. The E. coli KASII (FabF) was derived from the Gene Bank Accession Number P39435.

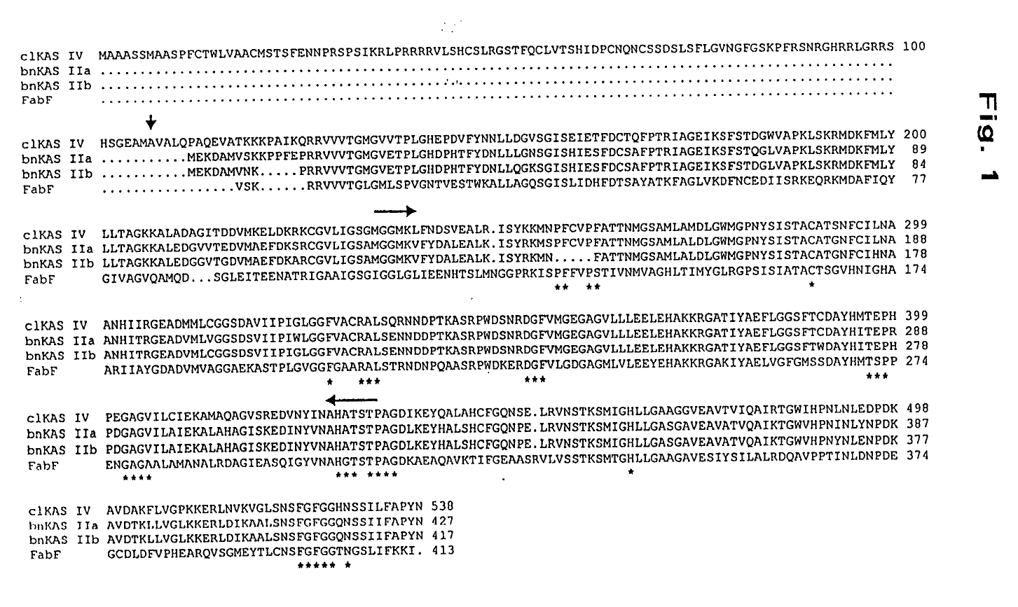

FIG. 2: Diagram for cloning clKAS4.

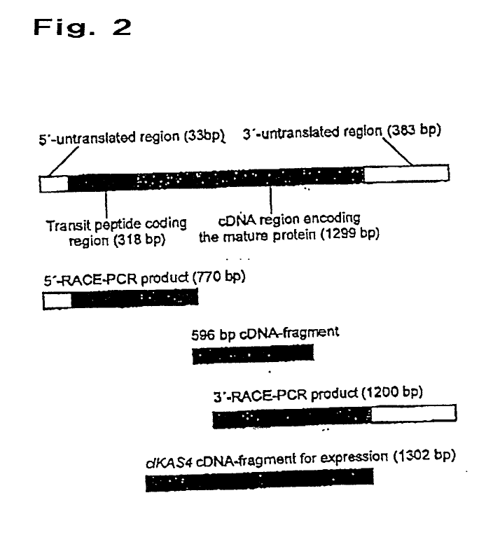

FIG. 3: Substrate specificity of the purified recombinant bnKASIIa (A) and clKASIV (B). The reaction products were separated by 2.5 M urea-PAGE, blotted on a PVDF membrane and visualized by autoradiography (upper portion of each of Figures A and B). The two bands of reaction products represent E. coli ACP isoforms such as those already observed previously (Winter et al. (1997) loc. cit.). The values show the mean=the standard deviation (n=4, for the substrate C₄n=2). Mal-ACP=malonyl-ACP; P-OH-ACP=β-hydroxyacyl-ACP.

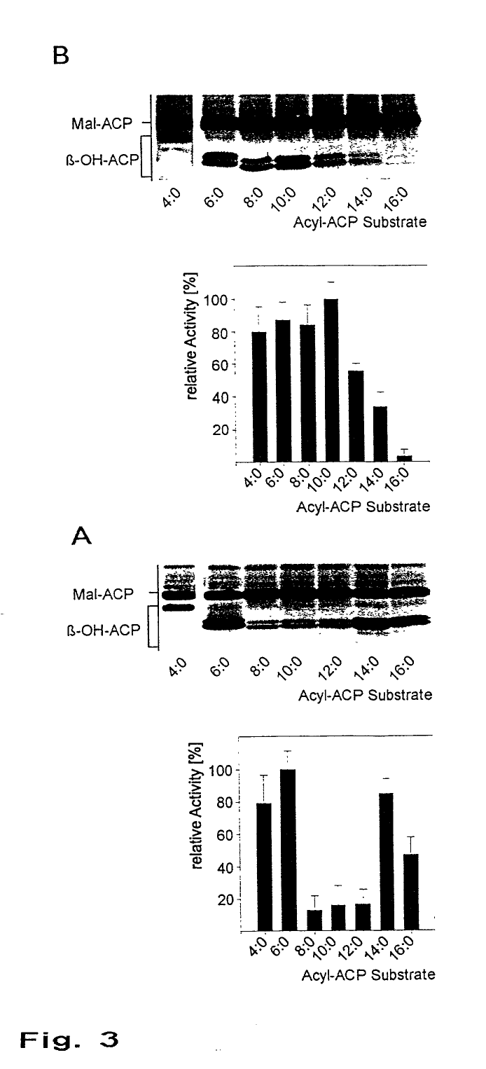

DNA and amino acid sequences for β-ketoacyl-ACP synthase (in 5′->3′ direction and from the N-terminal to the C-terminal amino acid, respectively).

```
1) SEQ ID:No. 1-β-ketoacyl-ACP synthase
      IV from Cuphea lanceolata
      DNA sequence of the cDNA clone clKAS4
```

CTACTTGGGTCGCCTCAGTTTTCAGGTGTTCCAATGGCGGCGGCCTCTTC
ATGGCTGCGTCACCGTTCTGTACGTGGCTCGTAGCTGCTTGCATGTCCAC
TTCCTTCGAAAACAACCCACGTTCGCCCTCCATCAAGCGTCTCCCCCGCC
GGAGGAGGGTTCTCTCCCATTGCTCCCTCCGTGGATCCACCTTCCAATGC
CTCGTCACCTCACACATCGACCCTTGCAATCAGAACTGCTCCTCCGACTC
CCTTAGCTTCATCGGGGTTAACGGATTCGGATCCAAGCCATTCCGGTCCA
ATCGCGGCCACCGGAGGCTCGGCCGTGCTTCCCATTCCGGGGAGGCCATG
GCTGTGGCTCTGCACCTGCACAGGAAGTCGCCACGAAGAAGAACCTGCTA
TCAAGCAAAGGCGAGTAGTTGTTACAGGAATGGGTGTGGTGACTCCTCTA
GGCCATGAACCTGATGTTTTCTACAACAATCTCCTAGATGGAGTAAGCGG
CATAAGTGAGATAGAGAACTTCGACAGCACTCAGTTTCCCACGAGAATTG
CCGGAGAGATCAAGTCTTTTTCCACAGATGGCTGGGTGGCCCCAAAGCTC
TCCAAGAGGATGGACAAGCTCATGCTTTACTTGTTGACTGCTGGCAAGAA
AGCATTAGCAGATGCTGGAATCACCGATGATGTGATGAAAGAGCTTGATA
AAAGAAAGTGTGGAGTTCTCATTGGCTCCGGAATGGGCGGCATGAAGTTG
TTCTACGATGCGCTTGAAGCCCTGAAAATCTCTTACAGGAAGATGAACCC
TTTTTGTGTACCTTTTGCCACCACAAATATGGGATCAGCTATGCTTGCAA
TGGATCTGGGATGGATGGGTCCAAACTACTCTATTTCAACTGCCTGTGCA
ACAAGTAATTTCTGTATACTGAATGCTGCAAACCACATAATCAGAGGCGA
AGCTGACATGATGCTTTGTGGTGGCTCGGATGCGGTCATTATACCTATCG
GTTTGGGAGGTTTTGTGGCGTGCCGAGCTTTGTCACAGAGGAATAATGAC
CCTACCAAAGCTTCGAGACCATGGGATAGTAATCGTGATGGATTTGTAAT
GGGCGAAGGAGCTGGAGTGTTACTTCTCGAGGAGTTAGAGCATGCAAAGA
AAAGAGGTGCAACCATTTATGCAGAATTTTTAGGGGGCAGTTTCACTTGC
GATGCCTACCACATGACCGAGCCTCACCCTGAGGAGCTGGAGTGATCCTC
TGCATAGAGAAGGCCATGGCTCAGGCCGGAGTCTCTAGAGAAGATGTAAA
TTACATAAATGCCCATGCAACTTCCACTCCTGCTGGAGATATCAAAGAAT
ACCAAGCTCTCGCCCACTGTTTCGGCCAAAACAGCGAGCTGAGAGTGAAT
TCCACTAAATCGATGATCGGTCATCTTCTTGGAGCAGCTGGTGGCGTAGA
AGCAGTTACTGTAATTCAGGCGATAAGGACTGGGTGGATCCATCCAAATC
TTAATTTGGAAGACCCGGACAAAGCCGTGGATGCAAAATTTCTCGTGGGA
CCTGAGAAGGAGAGACTGAATGTCAAGGTCGGTTTGTCCAATTCATTTGG
GTTCGGTGGGCATAACTCGTCTATACTCTTCGCCCCTTACAATTAGGTAT
GTTTCGTGTGGAATTCTTCGCTCAATGGATGCCAAAGTTTTTTAGAACTC
CTGCACGTTAGTAGCTTATGTCTCTGGACATGGAAATGGAATTTGGGTTG
GAAGCTGTAGCCAGAAGACTCAGAACCATGATAGACCGAGCACTCACGAC
GATGCCAAAGATACTCCTTGCCGGTATTGTTGTTAAGAGTCCNCTGTTTG
TCCCTTTTTTCTTTTCCTCTCTTCCTCATCGATATTAGTCGCACTTTTGA
GCTTTTGATCAAGCTAGTGAAGATACAAAGATACCTCGGGCACGTAGTTG
CTTGGTTTGCCACAATCTGTAAAACTCGGGACTGGTTTAGTTTCAGTGTG
TTTATCCTAAAAAAAAAAAAAAAAAAA

```
2) SEQ ID:No. 2-β-ketoacyl-ACP synthase
      IV from Cuphea lanceolata
      Amino acid sequence of the cDNA clone clKAS4
```

M A A A S S M A A S P F C T W L V A A
C M S T S F E N N P R S P S I K R L P
R R R R V L S H C S L R G S T F Q C L
V T S H I D P C N Q N C S S D S L S F
I G V N G F G S K P F R S N R G H R R
L G R A S H S G E A M A V A L Q P A Q
E V A T K K K P A I K Q R R V V V T G
M G V V T P L G H E P D V F Y N N L L
D G V S G I S E I E N F D S T Q F P T
R I A G E I K S F S T D G W V A P K L
S K R M D K L M L Y L L T A G K K A L
A D A G I T D D V M K E L D K R K C G
V L I G S G M G G M K L F Y D A L E A
L K I S Y R K M N P F C V P F A T T N
M G S A M L A M D L G W M G P N Y S I
S T A C A T S N F C I L N A A N H I I
R G E A D M M L C G G S D A V I I P I
G L G G F V A C R A L S Q R N N D P T
K A S R P W D S N R D G F V M G E G A
G V L L L E E L E H A K K R G A T I Y
A E F L G G S F T C D A Y H M T E P H
P E G A G V I L C I E K A M A Q A G V
S R E D V N Y I N A H A T S T P A G D
I K E Y Q A L A H C F G Q N S E L R V
N S T K S M I G H L L G A A G G V E A
V T V I Q A I R T G W I H P N L N L E
D P D K A V D A K F L V G P E K E R L
N V K V G L S N S F G F G G H N S S I
L F A P Y N

```
3) SEQ ID:No. 3-β-ketoacyl-ACP synthase
      II from Brassica napus
      DNA sequence of the cDNA clone bnKAS2a
```

ATGGAGAAGGATGCTATGGTTAGCAAGAAACCTCCTTTCGAGCCACGCCG
AGTTGTTGTCACTGGCATGGGAGTTGAAACGCCACTAGGTCACGACCCTC
ATACTTTTTATGACAACCTGCTTCTAGGCAACAGTGGTATAAGCCATATA
GAGAGTTTCGACTGTTCTGCATTTCCCACTAGAATCGCTGGAGAGATTAA
ATCTTTTTCGACCCAAGGATTGGTTGCTCCTAAACTTTCCAAAAGGATGG
ACAAGTTCATGCTTTACCTTCTCACCGCCGGCAAGAAGGCGTTGGAGGAT
GGTGTGGTGACTGAGGATGTGATGGCAGAGTTCGACAAATCAAGATGTGG
TGTCTTGATTGGCTCAGCAATGGGAGGCATGAAGGTCTTCTACGATGCGC
TTGAAGCTTTGAAAATCTCTTACAGGAAGATGAGCCCTTTTTGTGTACCT
TTTGCCACCACAAACATGGGTTCCGCTATGCTTGCCTTGGATCTGGGATG
GATGGGTCCAAACTACTCTATTTCAACCGCATGTGCCACGGGAAACTTCT
GTATTCTCAATGCAGCAAACCACATCACAAGAGGTGAAGCTGATGTAATG
CTCTGCGGTGGCTCTGACTCAGTTATTATTCCAATAGGGTTGGGAGGTTT
TGTTGCCTGCCGGGCTCTTTCAGAAAATAATGATGATCCCACCAAAGCTT
CTCGTCCTTGGGATAGTAACCGAGATGGTTTTGTTATGGGAGAGGGAGCC
GGAGTTCTACTTTTAGAAGAACTTGAGCATGCCAAGAAAAGAGGAGCAAC
TATATACGCAGAGTTCCTTGGGGGTAGTTTCACATGTGATGCATACCATA
TAACCGAACCACGTCCTGATGGTGCTGGTGTCATTCTCGCTATCGAGAAA
GCGTTAGCTCATGCCGGGATTTCTAAGGAAGACATAAATTACGTGAATGC
TCATGCTACCTCTACACCAGCTGGAGACCTTAAGGAGTACCACGCCCTTT
CTCACTGTTTTGGCCAAAATCCTGAGCTAAGGGTAAACTCAACAAAATCT
ATGATTGGACACTTGCTGGGAGCTTCTGGGGCCGTGGAGGCTGTTGCAAC
CGTTCAGGCAATAAAGACAGGATGGGTTCATCCAAATATCAACCTCGAGA
ATCCAGACAAAGCAGTGGATACAAAGCTTCTGGTGGGTCTTAAGAAGGAG
AGGCTGGATATCAAAGCAGCTTTGTCAAACTCTTTCGGCTTTGGTGGCCA
GAACTCTAGCATCATTTTCGCGCCCTACAACTGA

```
4) SEQ ID:No. 4-β-ketoacy1-ACP synthase
      II from Brassica napus
      Amino acid sequence of the cDNA clone bnKAS2a
```

M E K D A M V S K K P P F E P R R V V
V T G M G V E T P L G H D P H T F Y D
N L L L G N S G I S H I E S F D C S A
F P T R I A G E I K S F S T Q G L V A
P K L S K R M D K F M L Y L L T A G K
K A L E D G V V T E D V M A E F D K S
R C G V L I G S A M G G M K V F Y D A
L E A L K I S Y R K M S P F C V P F A
T T N M G S A M L A L D L G W M G P N
Y S I S T A C A T G N F C I L N A A N
H I T R G E A D V M L C G G S D S V I
I P I G L G G F V A C R A L S E N N D
D P T K A S R P W D S N R D G F V M G
E G A G V L L L E E L E H A K K R G A
T I Y A E F L G G S F T C D A Y H I T
E P R P D G A G V I L A I E K A L A H
A G I S K E D I N Y V N A H A T S T P
A G D L K E Y H A L S H C F G Q N P E
L R V N S T K S M I G H L L G A S G A
V E A V A T V Q A I K T G W V H P N I
N L E N P D K A V D T K L L V G L K K
E R L D I K A A L S N S F G F G G Q N
S S I I F A P Y N

```
5) SEQ ID:No. 5-β-ketoacyl-ACP synthase
      II from Brassica napus
      DNA sequence of the cDNA clone bnKAS2b
```

ATGGAGAAAGACGCCATGGTAAACAAGCCACGCCGAGTTGTTGTCACTGG
CATGGGAGTTGAAACACCACTAGGTCACGACCCTCATACTTTTTATGACA
ACTTGCTACAAGGCAAAAGTGGTATAAGCCATATAGAGAGTTTCGACTGT
TCTGCATTTCCCACTAGAATCGCTGGGGAGATTAAATCTTTTTCGACCGA
CGGATTGGTTGCTCCTAAACTTTCCAAAAGGATGGACAAGTTCATGCTCT
ACCTTCTAACAGCTGGCAAGAAGGCGTTGGAGGATGGTGGGGTGACTGGG
GATGTGATGGCAGAGTTCGACAAAGCAAGATGTGGTGTCTTGATTGGCTC
AGCAATGGGAGGCATGAAGGTCTTCTACGATGCGCTTGAAGCTTTGAAAA
TCTCTTACAGGAAGATGAATTTTGCCACCACAAACATGGGTTCCGCTATG
CTTGCCTTGGATCTGGGATGGATGGGTCCAAACTACTCTATTTCAACCGC
ATGTGCCACGGGAAACTTCTGTATTCACAATGCGGCAAACCACATTACTA
GAGGTGAAGCTGATGTAATGCTCTGTGGTGGCTCTGACTCAGTTATTATT
CCAATAGGGTTGGGAGGTTTTGTTGCCTGCCGGGCTCTTTCAGAAAATAA
TGATGATCCCACCAAAGCTTCTCGTCCTTGGGATAGTAACCGAGATGGTT
TTGTTATGGGAGAGGGAGCCGGAGTTCTACTTTTAGAAGAACTTGAGCAT
GCCAAGAAAAGAGGAGCAACTATATACGCAGAGTTCCTTGGGGGTAGTTT
CACATGGGATGCATATCATATTACCGAACCACATCCTGATGGTGCTGGTG
TCATTCTCGCTATCGAGAAAGCATTAGCTCATGCCGGGATTTCTAAGGAA
GACATAAATTACGTGAATGCTCATGCTACCTCTACACCAGCTGGAGACCT
TAAGGAGTACCACGCCCTTTCTCACTGTTTTGGCCAAAATCCTGAGCTAA
GGGTAAACTCAACAAAATCTATGATTGGACACTTGCTGGGAGCTTCTGGG
GCCGTGGAGGCTGTTGCAACCGTTCAGGCAATAAAGACAGGATGGGTTCA
TCCAAATTACAACCTCGAGAATCCAGACAAAGCAGTGGATACAAAGCTTC
TGGTGGGTCTTAAGAAGGAGAGACTGGATATCAAAGCAGCTTTGTCAAAC
TCTTTCGGCTTTGGTGGCCAGAACTCTAGCATCATTTTCGCCCCCTACAA
TTGA

```
6) SEQ ID:No. 6-β-ketoacyl-ACP synthase
      II from Brassica napus
      Amino acid sequence of the cDNA clone bnKAS2b
```

M E K D A M V N K P R R V V V T G M G
V E T P L G H D P H T F Y D N L L Q G
K S G I S H I E S F D C S A F P T R I
A G E I K S F S T D G L V A P K L S K
R M D K F M L Y L L T A G K K A L E D
G G V T G D V M A E F D K A R C G V L
I G S A M G G M K V F Y D A L E A L K
I S Y R K M N F A T T N M G S A M L A
L D L G W M G P N Y S I S T A C A T G
N F C I H N A A N H I T P G E A D V M
L C G G S D S V I I P I G L G G F V A
C R A L S E N N D D P T K A S R P W D
S N R D G F V M G E G A G V L L L E E
L E H A K K R G A T I Y A E F L G G S
F T W D A Y H I T E P H P D G A G V I
L A I E K A L A H A G I S K E D I N Y
V N A H A T S T P A G D L K E Y H A L
S H C F G Q N P E L R V N S T K S M I
G H L L G A S G A V E A V A T V Q A I
K T G W V H P N Y N L E N P D K A V D
T K L L V G L K K E R L D I K A A L S
N S F G F G G Q N S S I I F A P Y N

```
7) SEQ ID:No. 7-β-ketoacyl-ACP synthase
      I from Cuphea lanceolata
      DNA sequence of the cDNA clone clKAS 1
```

ACGATCTCAGCTCCAAAGCGCGAGTCCGACCCCAAGAAGCGTGTCGTCAT
CACCGGCATGGGCCTCGTCTCCATATTCGGATCCGACGTCGACGCCTACT
ACGACAAGCTGCTCTCCGGCGAGAGCGGCATCAGCTTAATCGACCGCTTC
GACGCTTCCAAGTTCCCCACCAGGTTCGGCGGCCAGATCCGTGGCTTCAA
CGCGACGGGCTACATCGACGGCAAGAACGACCGGCGGCTCGACGATTGCC
TCCGTTACTGCATTGTCGCCGGCAAGAAGGCTCTCGAAGACGCCGATCTC
GCCGGCCAATCCCTCTCCAAGATTGATAAGGAGAGGGCCGGAGTGCTAGT
TGGAACCGGTATGGGTGGCCTAACTGTCTTCTCTGACGGGGTTCAGAATC
TCATCGAGAAAGGTCACCGGAAGATCTCCCCGTTTTTCATTCCATATGCC
ATTACAAACATGGGGTCTGCCCTGCTTGCCATCGACTTGGGTCTGATGGG
CCCAAACTATTCGATTTCAACTGCATGTGCTACTTCCAACTACTGCTTTT
ATGCTGCTGCCAATCATATCCGCCGAGGTGAGGCTGACCTGATGATTGCT
GGACCAACTGAGGCTGCGATCATTCCAATTGGTTTAGGAGGATTCGTTGC
CTGCAGGGCTTTATCTCAAAGGAATGATGACCCTCAGACTGCCTCAAGGC
CGTGGGATAAGGACCGTGATGGTTTTGTGATGGGTGAAGGGGCTGGAGTA
TTGGTTATGGAGAGCTTGGAACATGCAATGAAACGGGGAGCGCCGATTAT
TGCAGAATATTTGGGAGGTGCAGTCAACTGTGATGCTTATCATATGACTG
ATCCAAGGGCTGATGGGCTTGGTGTCTCCTCATGCATTGAGAGCAGTCTC
GAAGATGCTGGGGTCTCACCTGAAGAGGTCAATTACATAAATGCTCATGC
GACTTCTACTCTTGCTGGGGATCTTGCCGAGATAAATGCCATCAAGAAGG
TTTTCAAGAACACCAAGGAAATCAAAATCAACGCAACTAAGTCAATGATC
GGCCACTGTCTTGGAGCATCAGGAGGTCTTGAAGCCATCGCAACCATTAA
GGGAATAACTTCCGGCTGGCTTCATCCCAGCATTAATCAATTCAATCCCG
AGCCATCGGTGGACTTCGACACTGTTGCCAACAAGAAGCAGCAACATGAA
GTCAACGTCGCTATCTCAAATTCATTCGGATTTGGAGGCCACAACTCAGT
TGTGGCTTTCTCAGCTTTCAAGCCATGA

```
8) SEQ ID:No. 8-β-ketoacyl-ACP synthase
      I from Cuphea lanceolata
      Amino acid sequence of the cDNA clone clKAS1
```

TISAPKRESDPKKRVVITGMGLVSIFGSDVDAYYDKLLSGESGISLIDRF
DASKFPTRFGGQIRGFNATGYIDGKNDRRLDDCLRYCIVAGKKALEDADL
AGQSLSKIDKERAGVLVGTGMGGLTVFSDGVQNLIEKGHRKISPFFIPYA
ITNMGSALLAIDLGLMGPNYSISTACATSNYCFYAAANHIRRGEADLMIA
GGTEAAIIPIGLGGFVACRALSQRNDDPQTASRPWDKDRDGFVMGEGAGV
LVMESLEHAMKRGAPIIAEYLGGAVNCDAYHMTDPRADGLGVSSCIESSL
EDAGVSPEEVNYINAHATSTLAGDLAEINAIKKVFKNTKEIKINATKSMI
GHCLGASGGLEAIATIKGITSGWLHPSINQFNPEPSVDFDTVANKKQQHE
VNVAISNSFGFGGHNSVVAFSAFKP

1. A nucleic acid sequence, characterized in that it encodes a protein with the activity of a β-ketoacyl-ACP synthase IV from Cuphea lanceolata.

2. A nucleic acid sequence according to claim 1, comprising SEQ:ID no. 1 or fragments thereof.

3. A nucleic acid sequence, characterized in that it encodes a protein with the activity of a β-ketoacyl-ACP-synthase II from Brassica napus.

4. A nucleic acid sequence according to claim 3, comprising SEQ:B) no. 3, SEQ:ID no. 5 or fragments thereof.

5. A nucleic acid sequence, characterized in that it encodes a protein with the activity of a β-ketoacyl-ACP-synthase I from Cuphea lanceolata.

6. A nucleic acid sequence according to claim 5, comprising SEQ:ID no. 7 or fragments thereof.

7. A nucleic acid molecule, characterized in that it includes a nucleic acid sequence according to one of the preceding claims.

8. A nucleic acid molecule according to claim 7, characterized in that it includes a nucleic acid sequence according to one of claims 1 to 6 in combination with a promoter that is active in plants.

9. A nucleic acid molecule according to claim 8, characterized in that the promoter is a promoter that is active in embryonal tissue.

10. A nucleic acid molecule according to one of claims 7 to 9, characterized in that it also contains enhancer sequences, sequences encoding signal peptides or other regulatory sequences.

11. A nucleic acid molecule according to one of claims 7 to 10, in which the coding nucleic acid sequence is present in the sense orientation.

12. A nucleic acid molecule according to one of claims 7 to 10, in which the coding nucleic acid sequence or parts thereof are present in the antisense orientation.

13. A protein with the enzymatic activity of a ,B-ketoacyl-ACP-synthase IV from Cuphea lanceolata.

14. A protein according to claim 13 which is coded by the sequence according to claim 2 or fragments thereof.

15. A protein with the enzymatic activity of a β-ketoacyl-ACP-synthase II from Brassica napus.

16. A protein according to claim 15, which is coded by the sequence according to claim 4 or fragments thereof.

17. A protein with the enzymatic activity of a β-ketoacyl-ACP-synthase I from Cuphea lanceolata.

18. A protein according to claim 17, which is coded by the sequence according to claim 6 or fragments thereof.

19. Transgenic plants containing a nucleic acid sequence or a nucleic acid molecule according to one of claims 1 to 12 as well as parts of these plants and their propagation material such as protoplasts, plant cells, callus, seeds, tubers or seedlings, etc. as well as the proengy of these plants.

20. Plants according to claim 19 having an altered fatty acid content in comparison with wild type plants and/or an altered fatty acid composition in comparison with wild-type plants.

21. Plants according to claim 19 or 20 having an increased medium-chain fatty acid content in comparison with wild-type plants.

22. Plants according to claim 19 or 20 having an increased short-chain fatty acid content in comparison with wild-type plants.

23. Plants according to claim 19 or 20 having an increased long-chain fatty acid content in comparison with wild-type plants.

24. Plants according to one of claims 19 to 23, additionally containing a nucleic acid sequence encoding a thioesterase, in particular a medium-chain-specific thioesterase or a short-chain-specific thioesterase.

25. Plants according to one of claims 19 to 24, wherein the plants are oil seed plants, in particular rape seed (Brassica napus), sunflower, soybeans, peanuts, coconut, turnip rape (Brassica rapa), cotton.

26. A method of increasing the medium-chain fatty acid content in plant seeds, comprising the steps:
a) preparing a nucleic acid sequence comprising at least the following components, which are aligned in 5′-3′ orientation:
a promoter which is active in plants, especially in embryonal tissue,
at least one nucleic acid sequence encoding a protein with the activity of a β-ketoacyl-ACP-synthase II or an active fragment thereof and
optionally a termination signal for termination of transcription and addition of a poly-A tail to the corresponding transcript, plus optionally DNA sequences derived therefrom;
b) transferring the nucleic acid sequence from a) to plant cells, and
c) optionally regenerating completely transformed plants and propagating the plants, if desired.

27. A method according to claim 26, in which the nucleic acid sequence encoding a protein with the activity of a β-ketoacyl-ACP-synthase IV or an active fragment thereof is a sequence according to claim 1 or 2.

28. A method according to claim 26 or 27, in which a nucleic acid sequence encoding a thioesterase, in particular a medium-chain-specific thioesterase, is additionally transferred.

29. A method of increasing the short-chain fatty acid content in plant seeds, comprising the steps:
a) preparing a nucleic acid sequence comprising at least the following components, which are aligned in 5′-3′ orientation:
a promoter which is active in plants, especially in embryonal tissue,
at least one nucleic acid sequence encoding a protein with the activity of a β-ketoacyl-ACP-synthase II or an active fragment thereof, and
optionally a termination signal for termination of transcription and addition of a poly-A tail to the corresponding transcript, plus optionally DNA sequences derived therefrom;
b) transferring the nucleic acid sequence from a) to plant cells, and
c) optionally regenerating completely transformed plants and reproducing the plants, if desired.

30. A method according to claim 29, wherein the nucleic acid sequence encoding a protein with the activity of a β-ketoacyl-ACP-synthase II or an active fragment thereof is a sequence according to claim 3 or 4.

31. A method according to claim 29 or 30, wherein the endogenous activity of β-ketoacyl-ACP-synthase I is also suppressed, e.g., by antisense or co-suppression.

32. A method according to one of claims 26 to 31, wherein a nucleic acid sequence encoding for thioesterase, in particular a medium-chain-specific or short-chain-specific thioesterase is also transferred.

33. A use of a plant produced according to one of claims 26 to 32 to produce vegetable oil with an increased fatty acid content.

34. A nucleic acid sequence, characterized in that it encodes a protein having the activity of a β-ketoacyl-ACP synthase II with substrate specificity for short- chain-acyl ACPs from Brassica napus.

35. A nucleic acid sequence according to claim 34, comprising SEQ:ID no.3, SEQ:ID no 5 or functionally active fragments thereof.

36. A nucleic acid molecule, characterized in that it comprises a nucleic acid sequence according to claim 34 or 35.

37. A nucleic acid sequence according to claim 36, characterized in that it comprises a nucleic acid sequence according to claim 34 or 35 in combination with a promoter that is active in plants.

38. A nucleic acid molecule according to claim 37, characterized in that the promoter is a promoter that is active in embryonal tissue.

39. A nucleic acid molecule according to one of claims 36 to 38, characterized in that it further contains enhancer sequences, sequences encoding signal peptides or other regulatory sequences.

40. A nucleic acid molecule according to one of claims 36 to 39, wherein the coding nucleic acid sequence is present in the sense orientation.

41. A nucleic acid molecule according to one of claims 36 to 39, wherein the coding nucleic acid sequence or functionally active fragments thereof are present in the anti sense orientation.

42. A protein having the enzymatic activity of a β-ketoacyl-ACP-synthase II with a substrate specificity for short-cain-acyl ACPs from Brassica napus.

43. A protein according to claim 42 which is coded by the sequence according to claim 35 or functionally active fragments thereof.

44. Transgenic plants containing a nucleic acid sequence or a nucleic acid molecule according to one of claims 34 to 45 as well as parts of these plants and their propagation material such as protoplasts, plant cells, callus, seeds, tubers or seedlings, etc. as well as the progeny of these plants

45. Plants according to claim 44 having an altered fatty acid content and/or an altered fatty acid composition in comparison with wild-type plants.

46. Plants according to claim 44 or 45 having an increased medium-chain fatty acid content in comparison with wild-type plants.

47. Plants according to claim 44 or 45 having an increased short-chain fatty acid content in comparison with wild-type plants.

48. Plants according to claim 44 or 45 having an increased long-chain fatty acid content in comparison with wild-type plants.

49. Plants according to one of claims 44 to 48, further containing a nucleic acid sequence encoding a thioesterase, in particular a medium-chain-specific thioesterase or a short-chain-specific thioesterase.

50. Plants according to one of claims 44 to 49, wherein the plants are oil seed plants, in particular rape seed (Brassica napus), sunflower, soybeans, peanuts, coconut, turnip rape (Brassica rapa), cotton.

51. A method of increasing the short-chain fatty acid content in plant seeds, comprising the steps of:
a) producing a nucleic acid sequence comprising at least the following components, which are in 5′-3′ orientation:
promoter which is active in plants, especially in embryonal tissue, at least one nucleic acid sequence encoding a protein having the activity of a 13-ketoacyl-ACP synthase II with substrate specificity for short-chain-acyl ACPS or a functionally active fragment thereof, and
optionally a termination signal for termination of transcription and addition of a poly-A tail to the corresponding transcript, as well as optionally DNA sequences derived therefrom;
b) transferring the nucleic acid sequence from a) to plant cells, and
c) optionally regenerating completely transformed plants and, if desired, propagating the plants.

52. A method according to claim 51, wherein the nucleic acid sequence encoding a protein with the activity of a β-ketoacyl-ACP synthase II or a functionally active fragment thereof is a sequence according to claim 34 or 35.

53. A method according to claim 51 or 52, wherein in addition the endogenous activity of the β-ketoacyl-ACP synthase I is suppressed, e.g. by antisense expression or co-suppression.

54. A method according to one of claims 51 to 53, wherein additionally a nucleic acid sequence encoding for thioesterase, in particular a medium-chain-specific or short-chain-specific thioesterase is transferred.

55. A use of a plant produced according to one of claims 51 to 54 for the production of vegetable oil having an increased fatty acid content.


SEQUENCE LISTING (16 sequences)

SEQ ID NO: 1 - DNA, 2031 bp, Cuphea lanceolata
  misc_feature (1)...(2031): n = A,T,C or G
ctacttgggtcgcctcagttttcaggtgttccaatggcggcggcctcttccatggctgcgtcaccgttctgtacgtggctcgtagctgcttgcatgtccacttccttcgaaaacaacccacgttcgccctccatcaagcgtctcccccgccggaggagggttctctcccattgctccctccgtggatccaccttccaatgcctcgtcacctcacacatcgacccttgcaatcagaactgctcctccgactcccttagcttcatcggggttaacggattcggatccaagccattccggtccaatcgcggccaccggaggctcggccgtgcttcccattccggggaggccatggctgtggctctgcaacctgcacaggaagtcgccacgaagaagaaacctgctatcaagcaaaggcgagtagttgttacaggaatgggtgtggtgactcctctaggccatgaacctgatgttttctacaacaatctcctagatggagtaagcggcataagtgagatagagaacttcgacagcactcagtttcccacgagaattgccggagagatcaagtctttttccacagatggctgggtggccccaaagctctccaagaggatggacaagctcatgctttacttgttgactgctggcaagaaagcattagcagatgctggaatcaccgatgatgtgatgaaagagcttgataaaagaaagtgtggagttctcattggctccggaatgggcggcatgaagttgttctacgatgcgcttgaagccctgaaaatctcttacaggaagatgaaccctttttgtgtaccttttgccaccacaaatatgggatcagctatgcttgcaatggatctgggatggatgggtccaaactactctatttcaactgcctgtgcaacaagtaatttctgtatactgaatgctgcaaaccacataatcagaggcgaagctgacatgatgctttgtggtggctcggatgcggtcattatacctatcggtttgggaggttttgtggcgtgccgagctttgtcacagaggaataatgaccctaccaaagcttcgagaccatgggatagtaatcgtgatggatttgtaatgggcgaaggagctggagtgttacttctcgaggagttagagcatgcaaagaaaagaggtgcaaccatttatgcagaatttttagggggcagtttcacttgcgatgcctaccacatgaccgagcctcaccctgaaggagctggagtgatcctctgcatagagaaggccatggctcaggccggagtctctagagaagatgtaaattacataaatgcccatgcaacttccactcctgctggagatatcaaagaataccaagctctcgcccactgtttcggccaaaacagcgagctgagagtgaattccactaaatcgatgatcggtcatcttcttggagcagctggtggcgtagaagcagttactgtaattcaggcgataaggactgggtggatccatccaaatcttaatttggaagacccggacaaagccgtggatgcaaaatttctcgtgggacctgagaaggagagactgaatgtcaaggtcggtttgtccaattcatttgggttcggtgggcataactcgtctatactcttcgccccttacaattaggtatgtttcgtgtggaattcttcgctcaatggatgccaaagttttttagaactcctgcacgttagtagcttatgtctctggacatggaaatggaatttgggttggaagctgtagccagaagactcagaaccatgatagaccgagcactcacgacgatgccaaagatactccttgccggtattgttgttaagagtccnctgtttgtcccttttttcttttcctctcttcctcatcgatattagtcgcacttttgagcttttgatcaagctagtgaagatacaaagatacctcgggcacgtagttgcttggtttgccacaatctgtaaaactcgggactggtttagtttcagtgtgtttatcctaaaaaaaaaaaaaaaaaaa

SEQ ID NO: 2 - protein, 538 aa, Cuphea lanceolata
MAAASSMAASPFCTWLVAACMSTSFENNPRSPSIKRLPRRRRVLSHCSLRGSTFQCLVTSHIDPCNQNCSSDSLSFIGVNGFGSKPFRSNRGHRRLGRASHSGEAMAVALQPAQEVATKKKPAIKQRRVVVTGMGVVTPLGHEPDVFYNNLLDGVSGISEIENFDSTQFPTRIAGEIKSFSTDGWVAPKLSKRMDKLMLYLLTAGKKALADAGITDDVMKELDKRKCGVLIGSGMGGMKLFYDALEALKISYRKMNPFCVPFATTNMGSAMLAMDLGWMGPNYSISTACATSNFCILNAANHIIRGEADMMLCGGSDAVIIPIGLGGFVACRALSQRNNDPTKASRPWDSNRDGFVMGEGAGVLLLEELEHAKKRGATIYAEFLGGSFTCDAYHMTEPHPEGAGVILCIEKAMAQAGVSREDVNYINAHATSTPAGDIKEYQALAHCFGQNSELRVNSTKSMIGHLLGAAGGVEAVTVIQAIRTGWIHPNLNLEDPDKAVDAKFLVGPEKERLNVKVGLSNSFGFGGHNSSILFAPYN

SEQ ID NO: 3 - DNA, 1284 bp, Brassica napus
atggagaaggatgctatggttagcaagaaacctcctttcgagccacgccgagttgttgtcactggcatgggagttgaaacgccactaggtcacgaccctcatactttttatgacaacctgcttctaggcaacagtggtataagccatatagagagtttcgactgttctgcatttcccactagaatcgctggagagattaaatctttttcgacccaaggattggttgctcctaaactttccaaaaggatggacaagttcatgctttaccttctcaccgccggcaagaaggcgttggaggatggtgtggtgactgaggatgtgatggcagagttcgacaaatcaagatgtggtgtcttgattggctcagcaatgggaggcatgaaggtcttctacgatgcgcttgaagctttgaaaatctcttacaggaagatgagccctttttgtgtaccttttgccaccacaaacatgggttccgctatgcttgccttggatctgggatggatgggtccaaactactctatttcaaccgcatgtgccacgggaaacttctgtattctcaatgcagcaaaccacatcacaagaggtgaagctgatgtaatgctctgcggtggctctgactcagttattattccaatagggttgggaggttttgttgcctgccgggctctttcagaaaataatgatgatcccaccaaagcttctcgtccttgggatagtaaccgagatggttttgttatgggagagggagccggagttctacttttagaagaacttgagcatgccaagaaaagaggagcaactatatacgcagagttccttgggggtagtttcacatgtgatgcataccatataaccgaaccacgtcctgatggtgctggtgtcattctcgctatcgagaaagcgttagctcatgccgggatttctaaggaagacataaattacgtgaatgctcatgctacctctacaccagctggagaccttaaggagtaccacgccctttctcactgttttggccaaaatcctgagctaagggtaaactcaacaaaatctatgattggacacttgctgggagcttctggggccgtggaggctgttgcaaccgttcaggcaataaagacaggatgggttcatccaaatatcaacctcgagaatccagacaaagcagtggatacaaagcttctggtgggtcttaagaaggagaggctggatatcaaagcagctttgtcaaactctttcggctttggtggccagaactctagcatcattttcgcgccctacaactga

SEQ ID NO: 4 - protein, 427 aa, Brassica napus
MEKDAMVSKKPPFEPRRVVVTGMGVETPLGHDPHTFYDNLLLGNSGISHIESFDCSAFPTRIAGEIKSFSTQGLVAPKLSKRMDKFMLYLLTAGKKALEDGVVTEDVMAEFDKSRCGVLIGSAMGGMKVFYDALEALKISYRKMSPFCVPFATTNMGSAMLALDLGWMGPNYSISTACATGNFCILNAANHITRGEADVMLCGGSDSVIIPIGLGGFVACRALSENNDDPTKASRPWDSNRDGFVMGEGAGVLLLEELEHAKKRGATIYAEFLGGSFTCDAYHITEPRPDGAGVILAIEKALAHAGISKEDINYVNAHATSTPAGDLKEYHALSHCFGQNPELRVNSTKSMIGHLLGASGAVEAVATVQAIKTGWVHPNINLENPDKAVDTKLLVGLKKERLDIKAALSNSFGFGGQNSSIIFAPYN

SEQ ID NO: 5 - DNA, 1254 bp, Brassica napus
atggagaaagacgccatggtaaacaagccacgccgagttgttgtcactggcatgggagttgaaacaccactaggtcacgaccctcatactttttatgacaacttgctacaaggcaaaagtggtataagccatatagagagtttcgactgttctgcatttcccactagaatcgctggggagattaaatctttttcgaccgacggattggttgctcctaaactttccaaaaggatggacaagttcatgctctaccttctaacagctggcaagaaggcgttggaggatggtggggtgactggggatgtgatggcagagttcgacaaagcaagatgtggtgtcttgattggctcagcaatgggaggcatgaaggtcttctacgatgcgcttgaagctttgaaaatctcttacaggaagatgaattttgccaccacaaacatgggttccgctatgcttgccttggatctgggatggatgggtccaaactactctatttcaaccgcatgtgccacgggaaacttctgtattcacaatgcggcaaaccacattactagaggtgaagctgatgtaatgctctgtggtggctctgactcagttattattccaatagggttgggaggttttgttgcctgccgggctctttcagaaaataatgatgatcccaccaaagcttctcgtccttgggatagtaaccgagatggttttgttatgggagagggagccggagttctacttttagaagaacttgagcatgccaagaaaagaggagcaactatatacgcagagttccttgggggtagtttcacatgggatgcatatcatattaccgaaccacatcctgatggtgctggtgtcattctcgctatcgagaaagcattagctcatgccgggatttctaaggaagacataaattacgtgaatgctcatgctacctctacaccagctggagaccttaaggagtaccacgccctttctcactgttttggccaaaatcctgagctaagggtaaactcaacaaaatctatgattggacacttgctgggagcttctggggccgtggaggctgttgcaaccgttcaggcaataaagacaggatgggttcatccaaattacaacctcgagaatccagacaaagcagtggatacaaagcttctggtgggtcttaagaaggagagactggatatcaaagcagctttgtcaaactctttcggctttggtggccagaactctagcatcattttcgccccctacaattga

SEQ ID NO: 6 - protein, 417 aa, Brassica napus
MEKDAMVNKPRRVVVTGMGVETPLGHDPHTFYDNLLQGKSGISHIESFDCSAFPTRIAGEIKSFSTDGLVAPKLSKRMDKFMLYLLTAGKKALEDGGVTGDVMAEFDKARCGVLIGSAMGGMKVFYDALEALKISYRKMNFATTNMGSAMLALDLGWMGPNYSISTACATGNFCIHNAANHITRGEADVMLCGGSDSVIIPIGLGGFVACRALSENNDDPTKASRPWDSNRDGFVMGEGAGVLLLEELEHAKKRGATIYAEFLGGSFTWDAYHITEPHPDGAGVILAIEKALAHAGISKEDINYVNAHATSTPAGDLKEYHALSHCFGQNPELRVNSTKSMIGHLLGASGAVEAVATVQAIKTGWVHPNYNLENPDKAVDTKLLVGLKKERLDIKAALSNSFGFGGQNSSIIFAPYN

SEQ ID NO: 7 - DNA, 1278 bp, Cuphea lanceolata
acgatctcagctccaaagcgcgagtccgaccccaagaagcgtgtcgtcatcaccggcatgggcctcgtctccatattcggatccgacgtcgacgcctactacgacaagctgctctccggcgagagcggcatcagcttaatcgaccgcttcgacgcttccaagttccccaccaggttcggcggccagatccgtggcttcaacgcgacgggctacatcgacggcaagaacgaccggcggctcgacgattgcctccgttactgcattgtcgccggcaagaaggctctcgaagacgccgatctcgccggccaatccctctccaagattgataaggagagggccggagtgctagttggaaccggtatgggtggcctaactgtcttctctgacggggttcagaatctcatcgagaaaggtcaccggaagatctccccgtttttcattccatatgccattacaaacatggggtctgccctgcttgccatcgacttgggtctgatgggcccaaactattcgatttcaactgcatgtgctacttccaactactgcttttatgctgctgccaatcatatccgccgaggtgaggctgacctgatgattgctggaggaactgaggctgcgatcattccaattggtttaggaggattcgttgcctgcagggctttatctcaaaggaatgatgaccctcagactgcctcaaggccgtgggataaggaccgtgatggttttgtgatgggtgaaggggctggagtattggttatggagagcttggaacatgcaatgaaacggggagcgccgattattgcagaatatttgggaggtgcagtcaactgtgatgcttatcatatgactgatccaagggctgatgggcttggtgtctcctcatgcattgagagcagtctcgaagatgctggggtctcacctgaagaggtcaattacataaatgctcatgcgacttctactcttgctggggatcttgccgagataaatgccatcaagaaggttttcaagaacaccaaggaaatcaaaatcaacgcaactaagtcaatgatcggccactgtcttggagcatcaggaggtcttgaagccatcgcaaccattaagggaataacttccggctggcttcatcccagcattaatcaattcaatcccgagccatcggtggacttcgacactgttgccaacaagaagcagcaacatgaagtcaacgtcgctatctcaaattcattcggatttggaggccacaactcagttgtggctttctcagctttcaagccatga

SEQ ID NO: 8 - protein, 425 aa, Cuphea lanceolata
TISAPKRESDPKKRVVITGMGLVSIFGSDVDAYYDKLLSGESGISLIDRFDASKFPTRFGGQIRGFNATGYIDGKNDRRLDDCLRYCIVAGKKALEDADLAGQSLSKIDKERAGVLVGTGMGGLTVFSDGVQNLIEKGHRKISPFFIPYAITNMGSALLAIDLGLMGPNYSISTACATSNYCFYAAANHIRRGEADLMIAGGTEAAIIPIGLGGFVACRALSQRNDDPQTASRPWDKDRDGFVMGEGAGVLVMESLEHAMKRGAPIIAEYLGGAVNCDAYHMTDPRADGLGVSSCIESSLEDAGVSPEEVNYINAHATSTLAGDLAEINAIKKVFKNTKEIKINATKSMIGHCLGASGGLEAIATIKGITSGWLHPSINQFNPEPSVDFDTVANKKQQHEVNVAISNSFGFGGHNSVVAFSAFKP

SEQ ID NO: 9 - protein, 413 aa, Escherichia coli
VSKRRVVVTGLGMLSPVGNTVESTWKALLAGQSGISLIDHFDTSAYATKFAGLVKDFNCEDIISRKEQRKMDAFIQYGIVAGVQAMQDSGLEITEENATRIGAAIGSGIGGLGLIEENHTSLMNGGPRKISPFFVPSTIVNMVAGHLTIMYGLRGPSISIATACTSGVHNIGHAARIIAYGDADVMVAGGAEKASTPLGVGGFGAARALSTRNDNPQAASRPWDKERDGFVLGDGAGMLVLEEYEHAKKRGAKIYAELVGFGMSSDAYHMTSPPENGAGAALAMANALRDAGIEASQIGYVNAHGTSTPAGDKAEAQAVKTIFGEAASRVLVSSTKSMTGHLLGAAGAVESIYSILALRDQAVPPTINLDNPDEGCDLDFVPHEARQVSGMEYTLCNSFGFGGTNGSLIFKKI

SEQ ID NO: 10 - DNA, 45 bp, Artificial Sequence
  Oligo-dT adapted primer
aactggaagaattcgcggccgcaggaatttttttttttttttttt

SEQ ID NO: 11 - DNA, 21 bp, Artificial Sequence
  A degenerate primer
atgggnggcagtgaaggtntt

SEQ ID NO: 12 - DNA, 20 bp, Artificial Sequence
  A degenerate primer
gtngangtngcatgngcatt

SEQ ID NO: 13 - protein, 7 aa, Brassica napus
MGGMKVF

SEQ ID NO: 14 - protein, 7 aa, Brassica napus
NAHATST

SEQ ID NO: 15 - DNA, 23 bp, Artificial Sequence
  A semi-specific oligonucleotide constructed with a 5′ NdeI restriction cleavage site based on the known sequences of B. rapa KAS II
catatggaraargaygcnatggt

SEQ ID NO: 16 - DNA, 20 bp, Artificial Sequence
  A semi-specific oligonucleotide  based on the known sequences of B. rapa KAS II
tcanttgtanggngcraaaa